(19) United States
(12) Patent Application Publication
(10) Pub. No.: US 2021/0043323 A1
(43) Pub. Date: Feb. 11, 2021

(54) Medical records and treatment system and process

(71) Applicants: Jonathan Lawrence Roden, Urbandale, IA (US); Khalid Boushaba, Elkridge, MD (US)

(72) Inventors: Jonathan Lawrence Roden, Urbandale, IA (US); Khalid Boushaba, Elkridge, MD (US)

(73) Assignees: Jonathan Lawrence Roden, Urbandale, IA (US); Khalid Lawrence Boushaba, Elkridge, MD (US)

(21) Appl. No.: 16/987,241

(22) Filed: Aug. 6, 2020

Related U.S. Application Data
(60) Provisional application No. 62/884,418, filed on Aug. 8, 2019.

(51) Int. Cl. G16H 50/20; G16H 10/20; G16H 10/60; G16H 50/70; G16H 40/20; G16H 40/67

(52) U.S. Cl. CPC G16H 50/20; G16H 10/20; G16H 10/60; A61B 5/024; G16H 40/20; G16H 40/67; G16H 50/70

(57) ABSTRACT

A method and system of providing patient self-screening of an encounter, taking of medical history, and refinement by medical professionals where patient medical information and prior encounters prior to a first stage with medical professionals are compared. Multiple issues are entered through a visual tablet based application. The medical information is passed between medical professionals and refined with input from the patient and at multiple stages and validated at each multiple stage.

### BACKGROUND OF THE INVENTION

The present invention is directed to a system and process of gathering medical information and determining treatments. More particularly, the invention is directed to a system and a process of collecting medical information through self-screening, medical history, and refinement for developing effective and efficient treatment of medical issues.

One of the most pressing issues in healthcare presently, and in the foreseeable future is the fact that there are more patients in the world than there are doctors who can provide personal healthcare for the patients. There are many silos in a medical environment that make tracking patient information between medical professionals difficult. Further, with current medical software there is a lack of transparency with respect to a timeline of a patient's treatment. Accordingly, a need exists in the art for a system that addresses these deficiencies.

### BRIEF SUMMARY OF THE INVENTION

The present invention addresses the above-described deficiencies as well as other deficiencies noted with prior art. In an aspect of present inventions, medical records and treatment system and process we are able to provide patients self-screening of encounter, medical history taking, and refinement by analytics, decision making, datamining, and medical professionals. By tracking and refinement at each step our invention can provide a better view of the patient's encounter, improve quality of treatment, and provide transparency for patients and medical professionals.

### BRIEF DESCRIPTION OF THE DRAWING

FIG. 1 is a conceptual diagram illustrating the workflow between the patients by analytics, decision making, datamining, and medical professionals that provides transparency, patient self-screening of encounter, medical history taking, and refinement by analytics, decision making, datamining, and medical professionals.

### DETAILED DESCRIPTION OF THE INVENTION

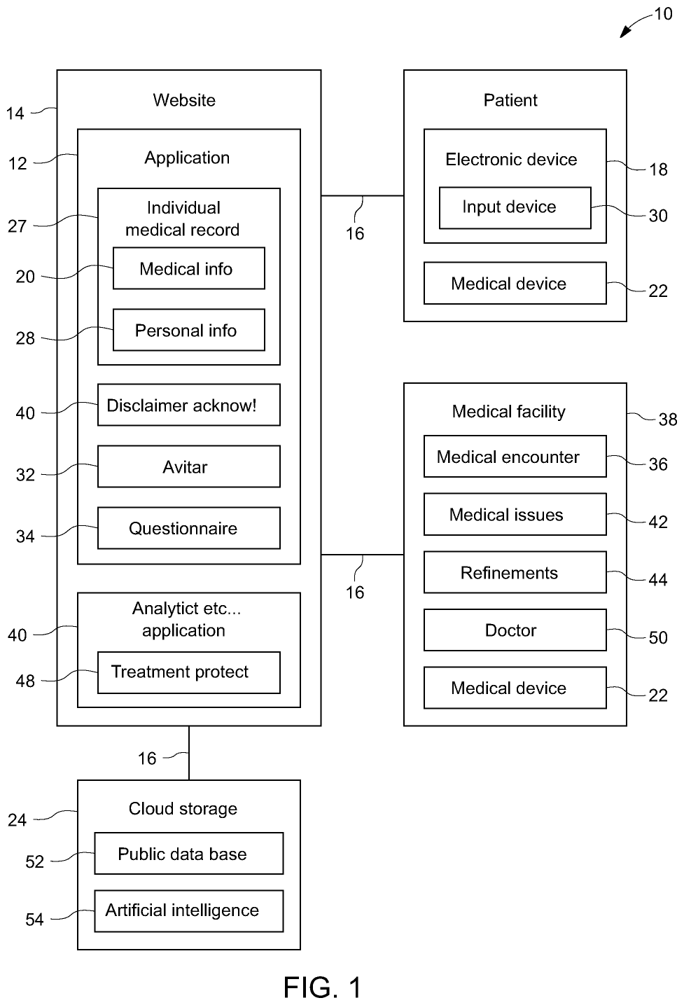

Referring to the FIG. 1, a medical records system 10 includes a downloadable medical records application 12 that is available from a website 14 via an electronic network 16. The medical records application 12 is downloaded to an electronic device 18 such as a personal computer, tablet, mobile phone or the like.

Once downloaded, the application is synched with and records medical information 20 from a medical device 22 such as a scale, a heart monitor, a fitness monitor, an EEG or EKG machine or the like. The application 12 is also linked to a secure cloud storage 24.

Initially, after downloading, a patient 26 establishes an individual medical record 27 by inputting personal information 28 about the individual and the individual's medical history. This personal information 28 is input using an input device 30 either by navigating the application 12 with an avatar 32 or responding to a questionnaire 34 provided by the application. The personal information 28 is stored in the individual medical record 27 on the secure cloud storage 24. Additional personal information 28 is received by the application 12 from the synced medical devices 22 and stored in the medical record 27 on the secured cloud 24.

Once a medical record 27 is established, the patient 26 starts a medical encounter 36 by contacting a medical facility 38 and scheduling an appointment. Alternatively, the medical record 27 is created at an initial medical encounter 36. The date and time of the appointment is added to the medical record 27.

The medical encounter 36 begins with the patient 26 retrieving, reviewing, updating and validating their medical record 27. The patient 26 also completes a HIPAA disclaimer acknowledgement 40 and authorizes medical facility 38 access to the patient's medical record 27.

Next, the patient 26 begins the intake step by completing a self-screening process. The self-screening process involves the patient 26 inputting information about medical issues 42. Information about medical issues 42 are input directly by the patient by navigating the application by using an avatar, and/or by answering a questionnaire. The patient 26 approves the medical issue information 42 which is added to the medical record 27.

Upon approval, a medical professional 43 reviews the updated medical record 27 and makes refinements 44. Refinements 44 are made based on an initial examination of the patient 26 and/or a review of the medical record 27. The refinements 44 are then provided to the patient 26 for review and validation that the refinements 44 are true and accurate to the best of the patient's knowledge and belief. Subsequent refinements 44 and patient validation are completed as needed.

Once approval has been provided for all refinements to the medial issue(s) 42, medical information and history 28, and refinements 44 are analyzed by an analytics, decision making, and datamining application 46 which determines a recommended treatment or protocol 48. The recommended treatment or protocol 48 is then reviewed by a medical doctor 50. The doctor 50 either refines the recommended treatment 48 for further analysis by the application 46 or approves the recommended treatment 48 which is provided to the patient 26 for final review and approval.

While information is maintained in individual medical files 27, information 28, 42, and 44 is pooled into a public database 52. This information in the public database 52 is available to medical facilities 38 and artificial intelligence 54 for analysis in developing more effective and/or efficient treatments and protocols.

1. Method for providing patient self-screening of encounter, medical history taking, and refinement by medical professionals, comprising the steps of:
comparing patient medical information and prior encounters prior to a first stage with medical professionals wherein patient medical information of multiple issues is entered through a visual tablet based application;
passing the medical information between medical professionals and refining with input from patient at multiple stages and validating at each multiple stage;
reviewing the refined medical information with patient and signing; and
analyzing the patient medical information, the prior encounters, and the refined medical information using analytics, decision making and data mining to recommend treatment to a patient for final review and approval.

2. The method of claim 1 further comprising the steps of self-screening of encounter by the patient and taking of medical history.

3. The method of claim 2 wherein the step of self-screening of encounter by the patient occurs in an emergency room setting.

4. The method of claim 3 further comprising the step of determining patient priority.
